(19) United States
(12) Patent Application Publication
(10) Pub. No.: US 2010/0174357 A1
(43) Pub. Date: Jul. 8, 2010

(54) Vascular Prosthesis of Varying Flexibility

(75) Inventors: George W. LeMaitre, Charlestown, MA (US); Robert L. Cannon, III, St. Germain de Coulamer (FR)

Correspondence Address: GREENBERG TRAURIG, LLP, ONE INTERNATIONAL PLACE, 20th FL, ATTN: PATENT ADMINISTRATOR, BOSTON, MA, 02110, US

(73) Assignee: LeMaitre Vascular, Inc.

(21) Appl. No.: 12/349,819

(22) Filed: Jan. 7, 2009

(51) Int. Cl. A61F 2/06; B32B 37/14

(52) U.S. Cl. 623/1.15; 623/1.13; 156/229

(57) ABSTRACT

A vascular prosthesis including a tubular body and a plurality of independent support members disposed on a surface of the tubular body. Spacing between the independent support members is varied to vary the flexibility of the vascular prosthesis.

### FIELD OF THE INVENTION

The invention relates generally to a vascular prosthesis of varying flexibility, and, in one embodiment, to an endovascular stent graft of varying flexibility.

### BACKGROUND OF THE INVENTION

Pressure and other factors can cause a weak area of a vessel (e.g., an aneurysm) to bulge, enlarge, and rupture. A stent graft, which is a tubular body supported by a support member such as a stent, can seal tightly with a vessel above and below a weak area. The stent graft is stronger than a weak area of a vessel, and can allow passage of blood through the vessel without stressing the weak area. Stent grafts can be used to treat a variety of conditions including abdominal and thoracic aortic aneurysms, as well as aneurysms in other locations.

Many stent grafts are rigid, which can reduce applicability or cause complications. In particular, rigid stent grafts have problems properly sealing in the aortic arch or in other highly curved regions. Flexible stent grafts can conform to vessel curvature, facilitating the treatment of curved vessels (e.g., a curve of a distal thoracic arch), but are less adapted to treatment of aneurysms in the descending aorta. When the pathology requires a prosthesis that extends from the aortic arch into the descending aorta there is, at present, no single solution. A desirable graft for this common pathology would comprise a flexible region adapted to the aortic arch and a more rigid region adapted to the descending aorta.

Typically, the flexibility of a stent or stent graft is modulated by varying a characteristic of the stent or stent graft. For example, the number of links or trusses coupling adjacent stents can be modulated while maintaining a constant stent spacing. Less coupling corresponds to a more flexible stent or stent graft. Truss spacing of individual wire stent pieces also can be varied. In other examples, the composition of the plastic or polymer material making up the graft is varied to modulate the flexibility of the stent graft.

### SUMMARY OF THE INVENTION

The invention, in various embodiments, features a vascular prosthesis of varying flexibility. A vascular prosthesis can include a tubular body and a plurality of independent support members (e.g., stents) disposed on a surface of the tubular body. Spacing between independent support members, among other features, can be varied to vary the flexibility of the vascular prosthesis. For example, a region of the vascular prosthesis can have support members spaced relatively closely or relatively distantly from one another. Increasing spacing between support members in a region of the vascular prosthesis generally increases flexibility of the region. Decreasing spacing between support members in a region of the vascular prosthesis generally decreases flexibility of the vascular prosthesis in that region.

A flexible prosthesis can conform to vasculature in which it is implanted. For example, a stent graft can exhibit sufficient flexibility to conform to vascular curvature, yet exhibit sufficient rigidity to remain straight in another section of the vasculature. A flexible prosthesis can improve treatment of vessels of small size, high curvature, or varying curvature (e.g., including regions of high and low curvature), and can minimize delivery-related complications. A rigid prosthesis can be used to repair an aneurysm if the anatomy permits it to seal properly the vessel. A prosthesis can be used to repair a vessel that has become narrow (e.g., stenosis of a coronary artery) or blocked (e.g., a plaque deposit).

In one aspect, the invention features a vascular prosthesis. The prosthesis includes a tubular body with a longitudinal axis. A plurality of independent support members are disposed on a surface of the tubular body. A first support member is spaced from a second support member along the longitudinal axis of the tubular body by a first support member spacing. The second support member is spaced from a third support member along the longitudinal axis of the tubular body by a second support member spacing. The first support member spacing is different than the second support member spacing.

In another aspect, the invention features a method of forming a vascular prosthesis. The method includes positioning a first support member on a surface of a tubular body, spacing a second support member on the surface of the tubular from the first support member by a first support member spacing, and spacing a third support member on the surface of the tubular body from the second support member by a second support member spacing. The first support member spacing is different than the second support member spacing.

In yet another aspect, the invention features a method of varying a flexibility of a vascular prosthesis. The method includes providing a tubular body having a longitudinal axis. A first support member is positioned on a surface of the tubular body. Next, a second support member is positioned on a surface of the tubular body, spaced from the first support member by a first support member spacing. The first support member and the second support member are spaced along a first portion of the longitudinal axis of the tubular body to form a first body region. A third support member is then spaced on the surface of the tubular body from the second support member by a second support member spacing. The second support member spacing is greater than the first support member spacing. The second support member and the third support member are spaced along a second portion of the longitudinal axis of the tubular body to form a second body region, which is more flexible than the first body region.

In still another aspect, a vascular prosthesis includes a substantially tubular body having a longitudinal axis. A plurality of independent support members are disposed on a surface of the tubular body. A first support member is associated with a first body region, and a second support member is associated with a second body region. A first feature of the first support member is different than a second feature of the second support member so that the second body region is more flexible than the first body region.

In other examples, any of the aspects above, or any apparatus or method described herein, can include one or more of the following features. The tubular body can be expanded polytetrafluoroethylene. In certain embodiments, an independent support member can be formed from a deformable material. In some embodiments, an independent support member is formed from a nickel-titanium alloy, stainless steel, or a shaped memory plastic.

In various embodiments, the vascular prosthesis can include a second tubular body and a plurality of independent support members disposed between the tubular body and the second tubular body. The plurality of independent support members, the tubular body, and the second tubular body can be sintered to form a laminate stent graft. The laminate stent graft can have a wall thickness of less than about 0.15 mm.

A vascular prosthesis can include a fourth support member spaced from the third support member along the longitudinal axis of the tubular body by the second support member spacing. The fourth support member can be linked to the third support member.

In some embodiments, the second support member spacing can be greater than the first support member spacing. The vascular prosthesis can include a first section of the tubular body with a first sub-set of the plurality of independent support members spaced at the first support member spacing. A second section of the tubular body can include a second sub-set of the plurality of independent support members spaced at the second support member spacing. The second section is more flexible than the first section.

In various embodiments, a vascular prosthesis can be formed by providing a plurality of independent support members, including the first support member, the second support member, and the third support member. A first sub-set of the plurality of independent support members are spaced in a first section of the tubular body at the first support member spacing. A second sub-set of the plurality of independent support members are spaced in a second section of the tubular body at the second support member spacing. The second section is more flexible than the first section.

In some embodiments, a vascular prosthesis can be formed by forming the tubular body with a first diameter from expanded polytetrafluoroethylene. The tubular body can be dilated to a second diameter by stretching the tubular body radially in small increments. The dilated tubular body can be sintered.

In various embodiments, a vascular prosthesis can be formed by placing the tubular body over a mandrel. A plurality of independent support members, including the first support member, the second support member, and the third support member are placed over the tubular body in a spaced fashion. A second tubular body having a diameter larger than the tubular body is placed over the plurality of independent support members and the tubular body. The plurality of independent support members, the tubular body, and the second tubular body are sintered to form a laminate stent graft. The laminate stent graft can be removed from the mandrel.

Features of the support members that can be varied to vary the flexibility of a vascular prosthesis include the diameter of the support member, the length of the support member, the number of apexes of the support member, the shape of the support member, the material of the support member, the composition of the support member, the material phase of the support member, the material phase of the region of the vascular prosthesis including the support member and a portion of the tubular body, and the spacing between adjacent support members. In certain embodiments, more than one feature of a single support member and/or different features of separate support members can be different to vary the flexibility of the vascular prosthesis, e.g., so that the second body region is more flexible than the first body region. In some embodiments, measurements of a blood vessel into which the vascular prosthesis is to be inserted are known so that a custom sized vascular prosthesis of varying flexibility can be formed.

The details of one or more examples are set forth in the accompanying drawings and the description below. Further features, aspects, and advantages of the invention will become apparent from the description, the drawings, and the claims.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1 shows a vascular prosthesis of varying flexibility including a tubular body and a plurality of support members.

FIG. 2 depicts engagement between adjacent support members.

FIG. 3 shows another vascular prosthesis including a tubular body and a plurality of support members disposed longitudinally on a surface of the tubular body with varying longitudinal spacings.

FIG. 4 shows yet another vascular prosthesis including a tubular body and a plurality of support members disposed longitudinally on a surface of the tubular body with varying longitudinal spacings.

FIG. 5 shows a cross section of an embodiment of a prosthesis where an independent support member is disposed on an outer surface of a tubular body.

FIG. 6 shows a cross section of an embodiment of a prosthesis where an independent support member is disposed on an inner surface of a tubular body.

FIG. 7 shows a cross section of an embodiment of a prosthesis where a second tubular body is placed over an independent support member disposed on an outer surface of a first tubular body.

FIG. 8 shows a vascular prosthesis including a support member that extends beyond a tubular body.

FIG. 9A is a flow diagram for an exemplary process for forming a vascular prosthesis.

FIG. 9B is a flow diagram for an exemplary process for forming a vascular prosthesis.

FIG. 9C is a flow diagram for an exemplary process for forming a vascular prosthesis.

FIG. 10 shows a support member.

### DETAILED DESCRIPTION OF THE INVENTION

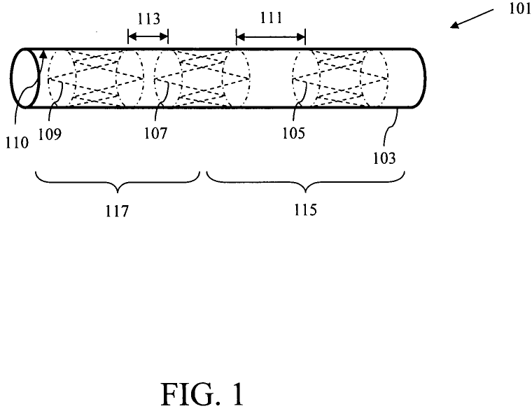

FIG. 1 shows an illustrative embodiment of a prosthesis 101 including a tubular body 103, a first independent support member 105, a second independent support member 107, and a third independent support member 109. The independent support members 105, 107, and 109 are disposed on a surface 110 of the tubular body 103. The first independent support member 105 is spaced from the second independent support member 107 by a first spacing 111. The third independent support member 109 is spaced from the second independent support member 107 by a second spacing 113. To vary flexibility of the prosthesis, the first spacing 111 can be different than the second spacing 113.

In some embodiments, the first spacing 111 is greater than the second spacing 113. In such an embodiment, the portion 115 of the prosthesis 101 including the first support member 105 and the second support member 107 is less flexible than the portion 117 of the prosthesis 101 including the second support member 107 and the third support member 109.

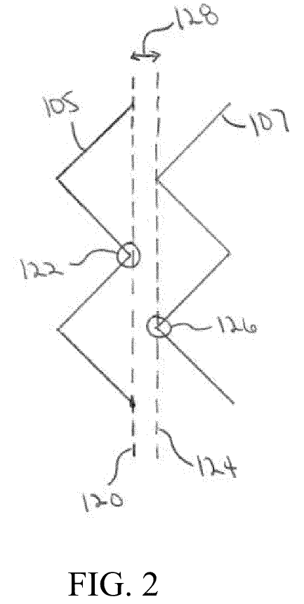

A support member can be formed as a circular band of material. The circular band can have a zigzag pattern, e.g., support members 105, 107, or 109 as shown in FIG. 1. Support member spacing, in general, can be described in terms of engagement of adjacent support members. Engagement represents the degree to which adjacent support members interface. FIG. 2 shows a side view of sections of adjacent support members 105 and 107. A first line 120 can be drawn through the apexes 122 on one edge of support member 105, and a second line 124 can be drawn through the apexes 126 on one edge of support member 105. The distance 128 between the first line 120 and the second line 124 can be referred to as the support member spacing.

In certain embodiments, the distance 128 can be between about minus 10 mm and about plus 10 mm, although larger or smaller distances can be used depending on the application. In some embodiments, the distance 128 can be between about zero mm and about plus 2 mm. For example, in FIG. 1, first spacing 111 can be plus 2 mm and second spacing 113 can be zero mm so that portion 115 is less flexible than portion 117.

In various embodiments, a support member can include a metal or metal alloy. In some embodiments, the metal alloy can be a nickel-titanium alloy or stainless steel. In one embodiment, the nickel-titanium alloy is Nitinol. In certain embodiments, a support member is formed from a shaped memory plastic. A support member can be a stent.

In various embodiments, the tubular body 103 can be formed from a polymeric material. For example, the polymeric material can be Dacron, Teflon, or Gore-Tex. In some embodiments, the polymeric material can be polytetrafluoroethylene (PTFE). In certain embodiments, the polytetrafluoroethylene is expandable or expanded polytetrafluoroethylene (ePTFE). In various embodiments, the tubular body 103 can be formed in an elliptical, conical, right cylindrical, tapered, cylindroid, or branched shape. The tubular body 103 can include an end that is beveled.

In various embodiments, between three and twenty support members can be positioned on a surface of a tubular body, although more or fewer can be used depending on the application. In some embodiments, between nine and twelve support members are used. In one embodiment, five support members are spaced at a first stent spacing and five support members are spaced at a second stent spacing.

In certain embodiments, a tubular body and a support member can be joined while both devices are in a compressed state. In other embodiment, a tubular body and a support member can be joined and then compressed to a compressed state. The formation of a prosthesis is can be considered complete when the prosthesis is in a compressed state.

In various embodiments, a tubular body can be affixed to an interior or exterior surface of a support member. In some embodiments, a tubular body can be affixed to the exterior surface of a support member, and a second tubular body can be affixed to the interior surface of the support member. The tubular body and the second tubular body can be joined together in the spaces between support members, the spaces within support members, and/or at the ends of the tubular bodies.

In certain embodiments, the tubular bodies can be joined together by an adhesive. In certain embodiments, the adhesive can be a polymer or thermoplastic fluoropolymer adhesive such as fluorinated ethylene propylene, perfluoroalkoxy, or polypropylene. In some embodiments, the tubular bodies can be joined together by heating above the crystalline melting point of the polymeric material to thermally adhere the tubular bodies. In various embodiments, a support member can be constrained between two tubular bodies, but permanently affixed to neither.

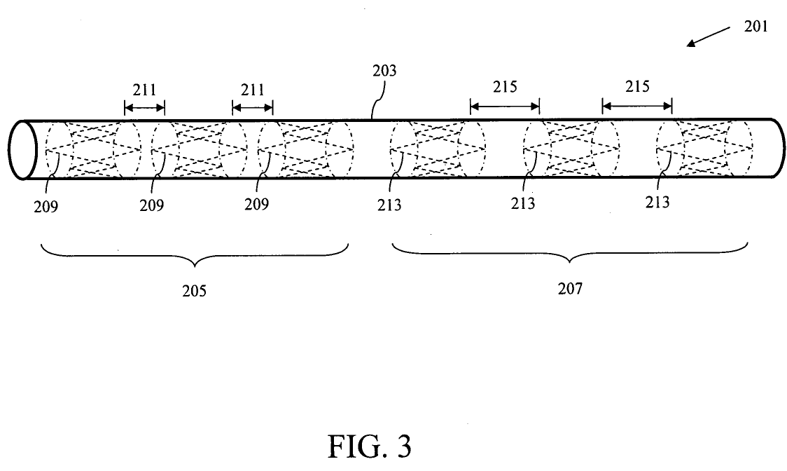

FIG. 3 shows an illustrative embodiment of a prosthesis 201 of varying flexibility. The prosthesis 201 includes a tubular body 203 with a first section 205 and a second section 207. The first section 205 includes a first plurality of independent support members 209, each spaced from an adjacent support member 209 by a first spacing 211. The second section 207 includes a second plurality of independent support members 213x, each spaced from an adjacent support member 213 by a second spacing 215. Because the second spacing 215 is greater than the first spacing 211, the second section 207 is less flexible than the first section 205.

In one embodiment, the prosthesis can be used to reinforce a portion of the vascular anatomy. For example, a thoracic device can include a top/proximal section more flexible than a bottom/distal section. The thoracic device can be used to repair a thoracic aortic aneurysm.

In certain embodiments, two or more support members can be interconnected in a vascular prosthesis of varying flexibility. For example, adjacent support members of the first plurality of independent support members 209 can be interconnected. In various embodiments, an apex of a first support member can be connected to an apex of a second support member.

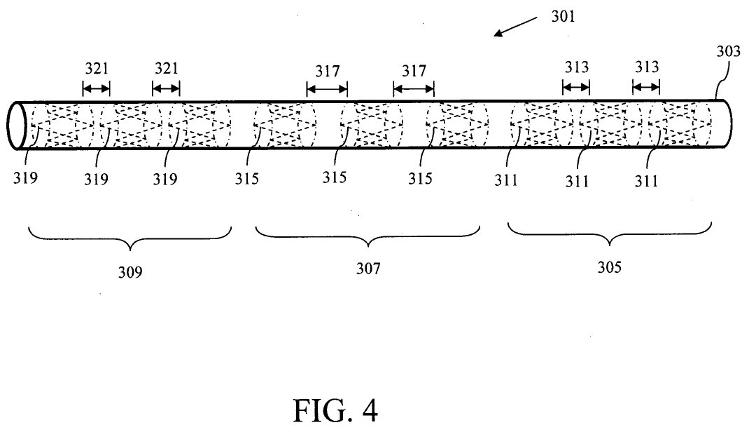

FIG. 4 shows an illustrative embodiment of a vascular prosthesis 301 of varying flexibility. The prosthesis 301 includes a tubular body 303 with a first section 305, a second section 307, and a third section 309. The first section 305 includes a first plurality of independent support members 311, each spaced from an adjacent support member 311 by a first spacing 313. The second section 307 includes a second plurality of independent support members 315, each spaced from an adjacent support member 315 by a second spacing 317. The third section 309 includes a third plurality of independent support members 319, each spaced from an adjacent support member 319 by a third spacing 321. Because the second spacing 317 is greater than the first spacing 313 and the third spacing 321, the second section 307 is less flexible than either of the first section 305 and the third section 309. In one embodiment, the first spacing 313 and the third spacing 321 are substantially the same.

In one embodiment, the prosthesis can be used to reinforce a portion of the vascular anatomy. For example, an abdominal device can include a middle section that is more flexible than a top/proximal section and a bottom/distal section. The abdominal device can be used to repair an abdominal aortic aneurysm.

In certain embodiments, adjacent support members can be linked. For example, referring to FIG. 4, two or more of support members 319 can be interconnected so that they are not independent. Second spacing 317 is different than third spacing 321 so that the flexibility of regions of the prosthesis 301 can be varied.

Prostheses, for example, as shown in FIGS. 1, 3 or 4 can include independent support members disposed on an outer surface of a tubular body, an inner surface of a tubular body, and/or between a first and second tubular body.

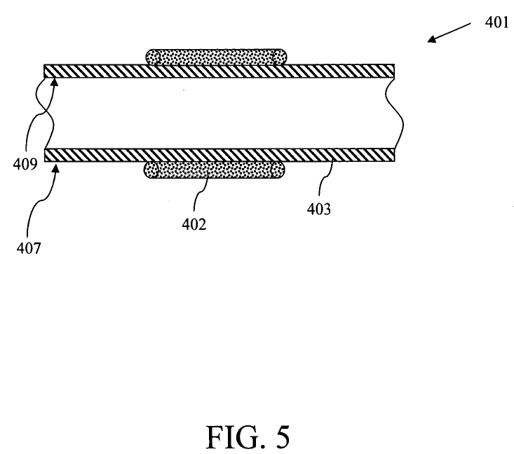

FIG. 5 presents a cross-section of an exemplary region 401 of a prosthesis. The region 401 includes a support member 402 and a tubular body 403 with an outer surface 407 and an inner surface 409. The support member 402 is disposed on the outer surface 407 of the tubular body 403.

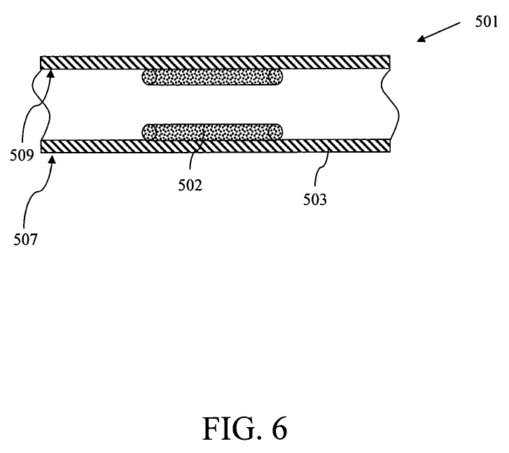

FIG. 6 presents a cross-section of an exemplary region 501 of a prosthesis. The region 501 includes a support member 502 and a tubular body 503 with an outer surface 507 and an inner surface 509. The support member 502 is disposed on the inner surface 509 of the tubular body 503.

In various embodiments, a prosthesis can be formed as a laminate stent graft. A plurality of independent support members can be disposed between two tubular bodies and sintered to form the laminate stent graft. In various embodiments, the laminate stent graft can have a wall thickness between about 0.1 mm to about 0.5 mm. In certain embodiments, the laminate stent graft has a wall thickness of less than about 0.15 mm.

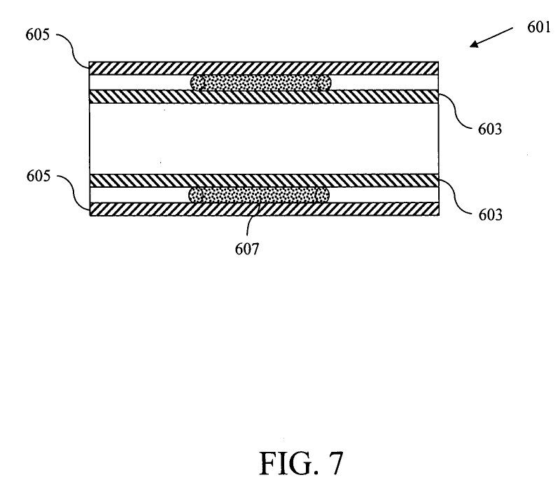

FIG. 7 shows a cross section of a region of a laminate stent graft 601. The laminate stent graft 601 includes a first tubular body 603, a second tubular body 605, and a support member 607. The support member 607 is laminated between the first tubular body 603 and the second tubular body 605.

In various embodiments, a sandwiched or laminate stent graft can include three or more layers. In one embodiment, a third layer is a middle layer or a liner. The middle layer or a liner can be formed from PTFE tape wrapped in a spiral pattern about the first tubular body.

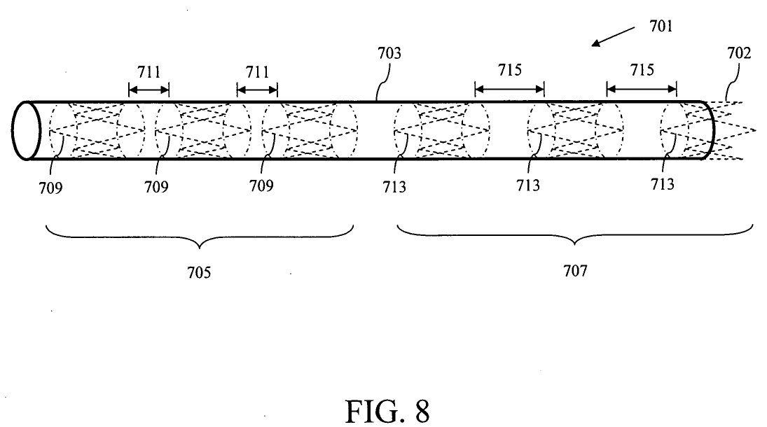

FIG. 8 shows an illustrative embodiment of a prosthesis 701 of varying flexibility including an open stent portion 702. The prosthesis 701 includes a tubular body 703 with a first section 705 and a second section 707. The first section 705 includes a first plurality of independent support members 709, each spaced from an adjacent support member 709 by a first spacing 711. The second section 707 includes a second plurality of independent support members 713, each spaced from an adjacent support member 713 by a second spacing 715. Because the second spacing 715 is greater than the first spacing 711, the second section 707 is less flexible than the first section 705. In another embodiment, the first spacing 711 is greater than the second spacing 715, and the first section 705 is less flexible than the second section 707.

The open stent portion 702 can provide vascular support while allowing blood flow through the open stent portion 702. The open stent portion 702 can also be positioned at a vascular branching to provide support while allowing blood flow through the open stent portion 702.

In various embodiments, a region of vasculature can include first and second regions, with the second region having greater curvature than the first region. A prosthesis, for example, as shown in FIGS. 1 or 3-8, can include first and second sections, with the second section having greater flexibility than the first section. The first section can assume the curvature of the first region and the second section can assume the curvature of the second region. Because flexibility of a section of a prosthesis can correspond to curvature of a region of vasculature, a prosthesis of varying flexibility can adapt to vasculature of varying curvature more readily than a prosthesis of substantially constant flexibility.

In various embodiments, prostheses such as those shown in FIGS. FIGS. 1 or 3-8, can be radially collapsible. For example, a stent graft can be radially collapsed and sheathed. In certain embodiments, the sheathed stent graft can be inserted into a patient's vasculature and positioned at a desired location. In certain embodiments, a catheter can be used to push the stent graft out of the sheath, or maintain the graft in place while the sheath is withdrawn. In one embodiment, the catheter is a flat ended catheter.

In various embodiments, a prosthesis can be biased to a cylindrical configuration by at least one stent. In some embodiments, when the prosthesis is unsheathed, it expands radially outward, and can thus engage an inner wall of a vessel. In other embodiments, a stent graft can be expanded by a balloon catheter.

In certain embodiments, a stent graft is selected so that its expanded outer diameter is larger than an inner diameter a vessel. In such embodiments, a force exerted by a stent can maintain a stent graft position. In one embodiment, a force exerted by a stent graft can maintain a stent graft position without suturing. In another embodiment, a stent graft can maintain a stent graft position with suturing.

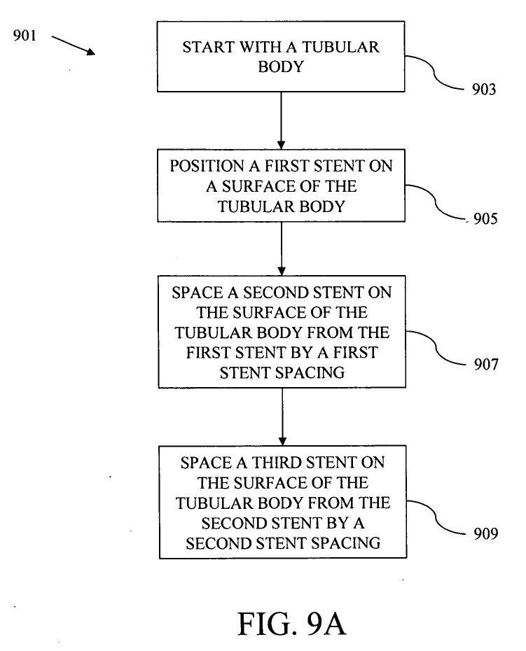

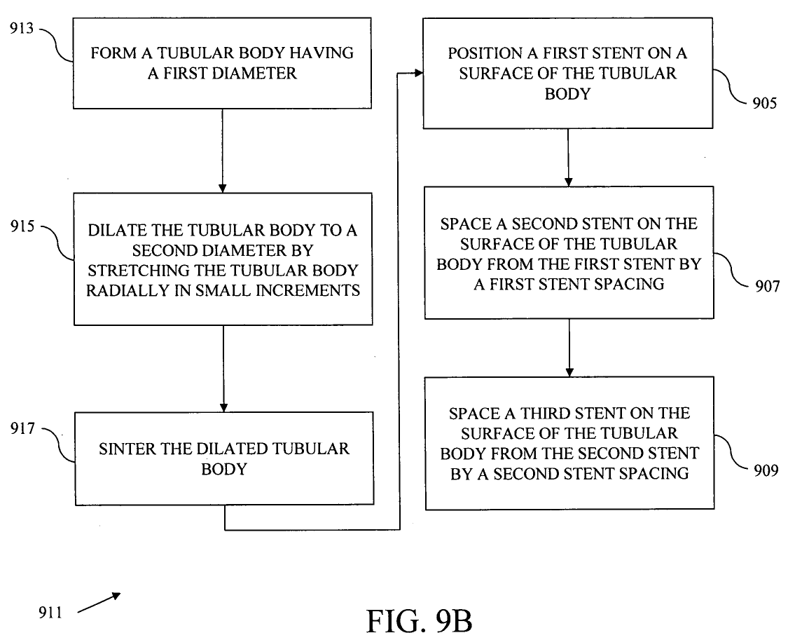

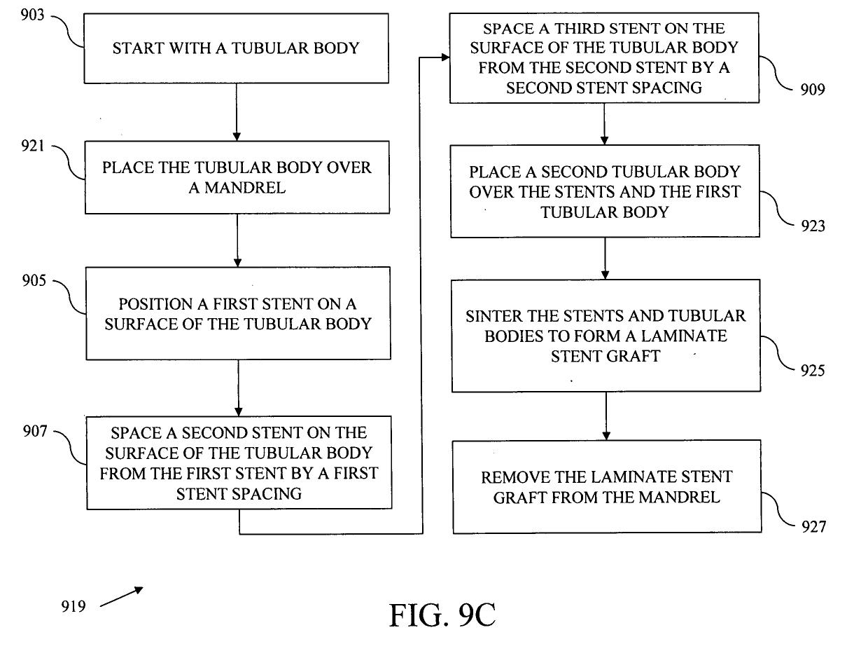

FIG. 9A presents a flow diagram 901 summarizing a process for forming a vascular prosthesis of varying flexibility. The process starts with a tubular body (903). A first support member is positioned on a surface of the tubular body (905). A second support member is spaced on the surface of the tubular from the first support member by a first support member spacing (907). A third support member is spaced on the surface of the tubular body from the second support member by a second support member spacing(909). The first support member spacing is different than the second support member spacing, resulting in the vascular prosthesis having varying flexibility.

FIG. 9B presents a flow diagram 911 summarizing a process for forming a vascular prosthesis of varying flexibility, including preparing the tubular body prior to spacing the support members in steps 905, 907, and 909. A tubular body having a first diameter is formed (913). The tubular body is dilated to a second diameter by stretching the tubular body radially in small increments (915). The dilated tubular body is sintered (917).

FIG. 9C presents a flow diagram 919 summarizing a process for forming a vascular prosthesis of varying flexibility, including preparing a laminate stent graft. The process starts with a tubular body (903), which is placed over a mandrel (921). The tubular body is prepared by spacing the support members in steps 905, 907, and 909. A second tubular body is placed over the support members and the first tubular body (923). The support members and the tubular bodies are sintered to form a laminate stent graft (925). The laminate stent graft is removed from the mandrel (927).

It is understood that all of the steps described in FIGS. 9A-9C need not be used to form a prosthesis of varying flexibility, and that in certain embodiments, one or more steps may need to be repeated. U.S. Pat. Nos. 6,187,054; 6,402,779; 6,443,981; and 6,605,119 describe exemplary processes for forming stent grafts, laminate stent grafts, and vascular prostheses and for radially expanding PTFE. These U.S. Patents are owned by the assignee of the instant application, and the entire disclosures are herein incorporated by reference.

In various embodiments, a stent graft can be customized to vasculature by measuring at least one dimension of a blood vessel and forming the stent graft to fit the vessel. Customizing size, shape, and/or flexibility of the stent graft can reduce the need for manufacturing or stocking stent grafts of numerous sizes. Suitable techniques for measuring the blood vessel include, but are not limited to, angiographical techniques, X-ray, computerized tomography, intravascular ultrasound, and spiral CT scan. In certain embodiments, a doctor can select at least one dimension. Dimensions measured can include a length, width, diameter, curvature, or shape (e.g., branching or bifurcation) of a region of vasculature.

U.S. patent application Ser. No. 09/644,640, the disclosure of which is herein incorporated by reference in its entirety, describes techniques for customizing stent grafts and vascular prostheses.

In various embodiment, only three dimensions are needed to produce a custom-sized prosthesis: a diameter of a lumen at which a first end of the intravascular device will be placed (e.g., the diameter of the lumen of the distal neck of an aorta), a diameter of a lumen at which a second end of the intravascular device will be placed (e.g., the diameter of the lumen of an iliac artery or the proximal neck of an aorta), and a length between the first end and the second end. In certain embodiments, other dimensions can be measured.

Other features of a support member can be varied to vary the flexibility of a vascular prosthesis. For example, the diameter of the material used to form the support member, the length of the support member, the number of apexes of the support member, and heat treatment received by a support member can be varied in the vascular prosthesis.

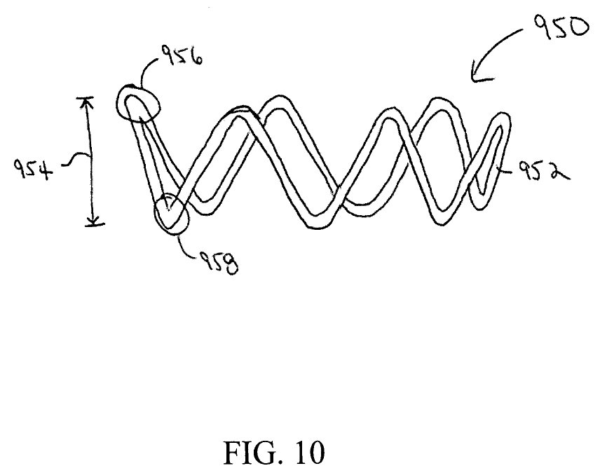

FIG. 10 shows a support member 950 formed from a circular band of wire 952. The diameter of the wire 952 can affect radial strength of the support member 950. Support member 950 can be used in the vascular prostheses shown in FIGS. 1 and 3-8. A larger diameter wire can result in a more rigid segment of a vascular prosthesis. Wire diameters, e.g., for nitinol, can be between about 0.016 inch and about 0.024 inch.

Longer support members result in a more rigid segment of a vascular prosthesis. As shown in FIG. 10, length 954 of the support member 950 is measured from an apex 956 on one side of a zigzag shaped support member to an apex 958 on the other side. Support members lengths, e.g., for a zigzag shaped support member, can be between about 0.5 cm and about 3 cm.

Using a support member with more apexes can result in a more flexible segment of a vascular prosthesis. The number of apexes, e.g., for a zigzag shaped support member, can be between 4 and 20. Support member 950 is shown with 12 apexes.

Differing stages of annealing, sintering, and/or heat treating a stent or stent graft can cause the support member material to be “at rest” in different material phases. Some phases are more flexible than others. The material phases can be selectively heated and selectively cooled so that the support member assumes a super-elastic phase or a more rigid phase.

In some embodiments, one or more of the features described herein can be varied to vary the flexibility of a vascular prosthesis. In addition, one or more features can be selected based on an examination of a blood vessel into which a vascular prosthesis is to be inserted. The vascular prosthesis can be formed to custom fit the blood vessel. Furthermore, one or more of the features of a support member or stent graft can be combined with measurements of a preselected blood vessel to form a customized vascular prosthesis of varying flexibility for the preselected blood vessel.

The invention has been described in terms of particular embodiments. The alternatives described herein are examples for illustration only and not to limit the alternatives in any way. The steps of the invention can be performed in a different order and still achieve desirable results. Other embodiments are within the scope of the following claims.

1. A vascular prosthesis comprising:
a substantially tubular body having a longitudinal axis; and
a plurality of independent support members disposed on a surface of the tubular body, a first support member being spaced from a second support member along the longitudinal axis of the tubular body by a first support member spacing and the second support member being spaced from a third support member along the longitudinal axis of the tubular body by a second support member spacing different than the first support member spacing.

2. The vascular prosthesis of claim 1 wherein the tubular body comprises expanded polytetrafluoroethylene.

3. The vascular prosthesis of claim 1 further comprising a second tubular body, the plurality of independent support members disposed between the tubular body and the second tubular body.

4. The vascular prosthesis of claim 3 wherein the plurality of independent support members, the tubular body, and the second tubular body are sintered to form a laminate stent graft.

5. The vascular prosthesis of claim 4 wherein the laminate stent graft has a wall thickness of less than about 0.15 mm.

6. The vascular prosthesis of claim 1 wherein each independent support member is comprised of a nickel-titanium alloy.

7. The vascular prosthesis of claim 1 wherein the second support member spacing is greater than the first support member spacing.

8. The vascular prosthesis of claim 7 wherein a first section of the tubular body comprises a first sub-set of the plurality of independent support members spaced at the first support member spacing and a second section of the tubular body comprises a second sub-set of the plurality of independent support members spaced at the second support member spacing, the second section being less flexible than the first section.

9. The vascular prosthesis of claim 1 further comprising a fourth support member spaced from the third support member along the longitudinal axis of the tubular body by the second support member spacing, the fourth support member linked to the third support member.

10. A method of forming a vascular prosthesis comprising:
positioning a first support member on a surface of a tubular body;
spacing a second support member on the surface of the tubular from the first support member by a first support member spacing; and
spacing a third support member on the surface of the tubular body from the second support member by a second support member spacing different than the first support member spacing to form the vascular prosthesis.

11. The method of claim 10 further comprising:
forming the tubular body from expanded polytetrafluoroethylene, the tubular body having a first diameter;
dilating the tubular body to a second diameter by stretching the tubular body radially in small increments; and
sintering the dilated tubular body.

12. The method of claim 10 further comprising:
placing the tubular body over a mandrel;
placing a plurality of independent support members, including the first support member, the second support member, and the third support member, over the tubular body in a spaced fashion;
placing a second tubular body having a diameter larger than the tubular body over the plurality of independent support members and the tubular body; and
sintering the plurality of independent support members, the tubular body, and the second tubular body to form a laminate stent graft.

13. The method of claim 10 wherein the second support member spacing is greater than the first support member spacing.

14. The method of claim 13 further comprising:
providing a plurality of independent support members, including the first support member, the second support member, and the third support member;
spacing in a first section of the tubular body a first sub-set of the plurality of independent support members at the first support member spacing; and
spacing in a second section of the tubular body a second sub-set of the plurality of independent support members at the second support member spacing, the second section being less flexible than the first section.

15. A method of varying flexibility of a vascular prosthesis comprising:
providing a tubular body having a longitudinal axis;
positioning a first support member on a surface of the tubular body;
spacing on the surface of the tubular body a second support member from the first support member by a first support member spacing, the first support member and the second support member being spaced along a first portion of the longitudinal axis of the tubular body to form a first body region; and
spacing on the surface of the tubular body a third support member from the second support member by a second support member spacing greater than the first support member spacing, the second support member and the third support member being spaced along a second portion of the longitudinal axis of the tubular body to form a second body region less flexible than the first body region of the vascular prosthesis.

16. The method of claim 15 further comprising:
forming the tubular body from expanded polytetrafluoroethylene, the tubular body having a first diameter;
dilating the tubular body to a second diameter by stretching the tubular body radially in small increments; and
sintering the dilated tubular body.

17. The method of claim 15 further comprising:
placing the tubular body over a mandrel;
placing a plurality of independent support members, including the first support member, the second support member, and the third support member, over the tubular body and along the longitudinal axis in a spaced fashion;
placing a second tubular body having a diameter larger than the tubular body over the plurality of independent support members and the tubular body; and
sintering the plurality of independent support members, the tubular body, and the second tubular body to form a laminate stent graft.

18. The method of claim 15 further comprising:
providing a plurality of independent support members, including the first support member, the second support member, and the third support member;
spacing in a first section of the tubular body a first sub-set of the plurality of independent support members at the first support member spacing; and
spacing in a second section of the tubular body a second sub-set of the plurality of independent support members at the second support member spacing, the second section being more flexible than the first section.

19. A vascular prosthesis comprising:
a substantially tubular body having a longitudinal axis; and
a plurality of independent support members disposed on a surface of the tubular body, a first support member associated with a first body region and a second support member associated with a second body region, a first feature of the first support member different than a second feature of the second support member so that the second body region is more flexible than the first body region.

20. The vascular prosthesis of claim 19 wherein the first feature and the second feature represent the diameter of the wire of the respective support member.

21. The vascular prosthesis of claim 19 wherein the first feature and the second feature represent the length of the wire of the respective support member.

22. The vascular prosthesis of claim 19 wherein the first feature and the second feature represent the number of apexes of the respective support member.

23. The vascular prosthesis of claim 19 wherein the first feature and the second feature represent the spacing between the respective support member and an adjacent support member.

24. The vascular prosthesis of claim 19 wherein the first feature and the second feature represent the shape of the respective support member.

25. The vascular prosthesis of claim 19 wherein the first feature and the second feature represent the material phase of the respective support member.

26. The vascular prosthesis of claim 19 wherein more than one feature of each of the first support member and the second support member are different so that the second body region is more flexible than the first body region.

27. The vascular prosthesis of claim 19 wherein measurements of a blood vessel into which the vascular prosthesis is to be inserted are known so that a custom sized vascular prosthesis of varying flexibility can be formed.
